(19) United States
(12) Patent Application Publication
(10) Pub. No.: US 2007/0043254 A1
(43) Pub. Date: Feb. 22, 2007

(54) Underwear Having Detachable Magnetic Devices

(76) Inventor: Jill R. DeMarco, Arnold, MD (US)

Correspondence Address: MEDLER FERRO PLLC, 8607 ROCKDALE LANE, SPRINGFIELD, VA, 22153, US

(21) Appl. No.: 11/465,526

(22) Filed: Aug. 18, 2006

Related U.S. Application Data
(60) Provisional application No. 60/711,036, filed on Aug. 18, 2005.

(51) Int. Cl. A61N 1/00

(52) U.S. Cl. 600/15

(57) ABSTRACT

An undergarment for applying a magnetic field to a wearer for therapy, such as for pain management due to menstrual cramping in a female. The undergarment being underwear having leg openings and a band that defines a waist opening. The underwear include an aperture adjacent or proximate to the band for receiving a detachable magnetic device. Alternatively, the underwear may include a ring-shaped connector for receiving the detachable magnetic device. The detachable magnetic device has a pair of magnets for creating the magnetic field with a flexible strap portion that holds one of the pair of magnets at each end thereof. The magnetic device is attachable to the underwear at the aperture or ring-shaped connector by attraction between the pair of magnets.

### CROSS-REFERENCE TO RELATED APPLICATION

This application claims the benefit of U.S. Provisional Application No. 60/711,036 filed Aug. 18, 2005, which is incorporated by reference herein in its entirety.

### FIELD OF THE INVENTION

The present invention relates to underwear to be worn by a female, and more particularly, to underwear having attachable magnets for applying a magnetic field to the body for natural pain management.

### BACKGROUND OF THE INVENTION

Magnetic treatment for the reduction of pain has been used for centuries all over the world. Magnets are considered by some to be a natural means of treating body pains and ailments, and many methods and mechanisms have been used to apply magnets to various body areas to ease pain or soreness. Recent research suggests that, at least in some cases, topical application of permanent magnets may indeed be useful in pain relief. Although theories abound, the mechanism for such an effect remains unexplained.

Various belt/band and clip-on devices are known for placing a magnet or magnets in topical contact with an area of the body to be treated. However, such devices are often uncomfortable and/or bulky, particularly under clothing, and are not well suited for relief of pain or muscle soreness in the lower back and abdominal regions of the body. Various male undergarments are known with magnetic strips sewn into or proximate a groin area for relief of penile problems. As well, magnetic fabric sheets for use in making various garments, such as underwear, are known. However, garments having permanently attached magnets or that are made from magnetic material do not offer flexibility in re-positioning of the magnets or magnetic material, and may not otherwise be worn or laundered without the magnets or magnetic material being attached.

Thus a need exists for an undergarment that includes one or more unobtrusive magnetic devices for relief of pain and/or muscle soreness in the lower back and/or abdominal regions of the body, wherein the magnetic devices are detachable and/or repositionable with respect to the undergarment.

### BRIEF SUMMARY OF THE INVENTION

The present invention is directed to an undergarment for applying a magnetic field to a wearer for therapy, such as for pain management of menstrual cramping in a female. In an embodiment, the undergarment is underpants having leg openings and a band that defines a waist opening. The underpants include at least one aperture adjacent or proximate to the band. The undergarment further includes a detachable magnetic device that has a pair of magnets for creating the magnetic field. The magnetic device includes a flexible strap portion that holds a magnet at each end thereof. The magnetic device is attachable to and detachable from the underpants at the aperture by attraction between the pair of magnets.

In another embodiment, an undergarment for applying a magnetic field to a wearer for therapy includes underpants having leg openings and a band defining a waist opening, wherein the band includes at least one ring-shaped connector attached thereto. The undergarment further includes a detachable magnetic device housing a pair of magnets for creating the magnetic field. The magnetic device includes a strap portion for holding the magnets, such that a magnet is positioned at each end of the strap portion of the magnetic device. The magnetic device is attachable to and detachable from the underpants at the ring-shaped connector by attraction between the pair of magnets.

In further embodiments, the undergarment may include a plurality of apertures or ring-shaped connectors with one or more of the apertures or connectors having a detachable magnetic device attached thereto. Underpants or underwear in accordance with various embodiments of the present invention may be any of a brief, bikini, hiphugger, boyshort or thong-style, as may be worn by a male or female.

### BRIEF DESCRIPTION OF DRAWINGS

The foregoing and other features and advantages of the invention will be apparent from the following description of the invention as illustrated in the accompanying drawings. The accompanying drawings, which are incorporated herein and form a part of the specification, further serve to explain the principles of the invention and to enable a person skilled in the pertinent art to make and use the invention. The drawings are not to scale.

FIG. 1 illustrates a front view of an undergarment having a magnetic device in accordance with an embodiment of the present invention.

FIG. 2 illustrates a front view of an undergarment having a magnetic device in accordance with another embodiment of the present invention.

FIGS. 3 and 3A illustrate a top and side view of the magnetic device shown in FIG. 1 in an open position.

FIGS. 4 and 4A illustrate a top and side view of the magnetic device shown in FIG. 2 in an open position.

FIG. 5 is an enlarged view of a band portion of an undergarment in accordance with another embodiment of the present invention.

FIG. 6 illustrates an alternative side view of the magnetic device shown in FIG. 2 in an open position.

### DETAILED DESCRIPTION OF THE INVENTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

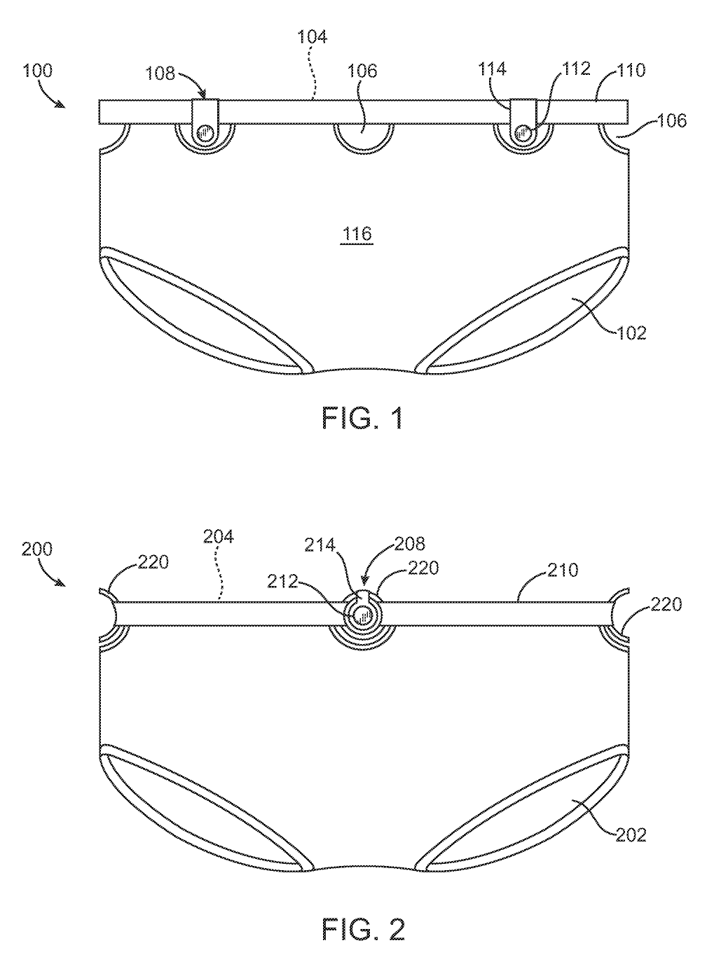

FIG. 1 illustrates a front view of an undergarment having a magnetic device in accordance with an embodiment of the present invention. Underpants 100 have leg openings 102 and a waist opening 104 that is defined by a band 110. The legs of a wearer pass through leg openings 102 such that waist opening 104 encircles the wearer at, near or below the waistline depending on the particular style of the underpants and/or the fit on the wearer. Underpants 100 include apertures 106 situated below band 110 and spaced around the front, back and sides of the undergarment.

In the embodiment of FIG. 1, detachable magnetic devices 108 are shown encircling band 110 at two apertures 106. In various embodiments, one or more magnetic devices 108 may be utilized in one or more apertures 106 for pain management as may be desired by the wearer.

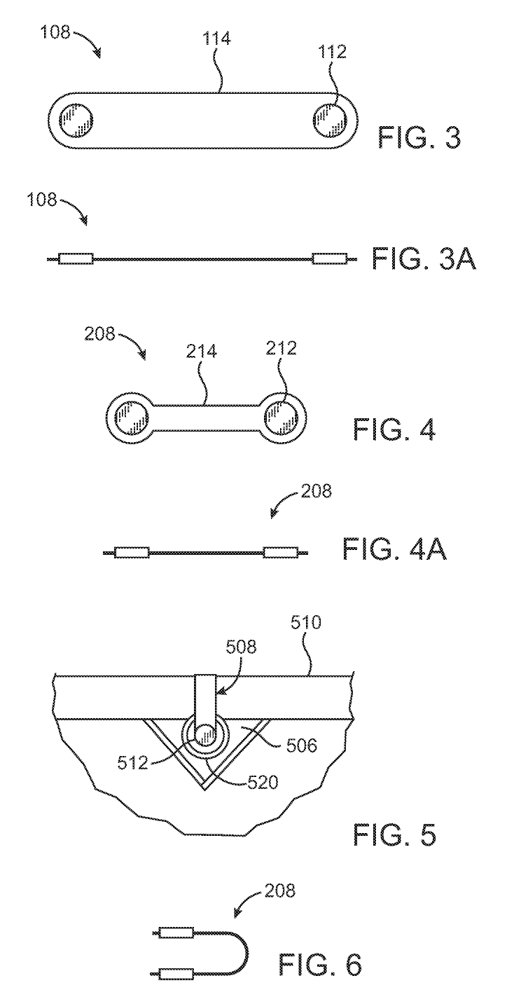

As shown in FIGS. 3 and 3A, which illustrate a top and side view of the magnetic device shown in FIG. 1, magnetic device 108 includes a pair of polarized magnets 112 that are used to create the therapeutic magnetic field. Magnetic device 108 further includes a flexible strap portion 114 with one of the pair of magnets 112 positioned and secured at either end thereof. In an embodiment, strap portion 114 is of thermoplastic polyurethane that is molded over magnets 112 to secure the magnets therein and to provide a thin, unobtrusive profile to magnetic device 108.

Magnetic device 108 is attached at a respective aperture 106 by attraction between the pair of magnets 112. Flexible strap portion 114 of magnetic device 108 encircles band 110 and situates magnets 112 within aperture 106 when magnetic device 108 is attached to underpants 100. Strap portion 114 also aids in maintaining magnetic device 108 in position proximate aperture 106 during normal wear. Magnetic device 108 is easily detached from aperture 106 through the separation of magnets 112, and is repositionable to another aperture 106, if so desired.

In another embodiment, apertures 106 may be in the form of a slit or button-hole in the material that forms a body portion 116 of underpants 100. The slit or slits may be made anywhere on the undergarment, i.e., within a front, back and/or side panel, that would be beneficial for placing magnetic device 108 in contact with an area of the wearer to be treated. In such an embodiment, when magnetic device 108 is attached to underpants 100, strap portion 114 of magnetic device 108 straddles the slit such that one of the pair of magnets 112 is positioned against an interior surface of underpants 100 in contact with the wearer, and the other of the pair of magnets 112 is positioned against an exterior surface of underpants 100. In such an embodiment, the magnet which is placed next to the wearer's skin may be stronger, i.e., of a higher Gauss measurement, than the exterior magnet.

FIG. 2 illustrates a front view of an undergarment having a magnetic device in accordance with another embodiment of the present invention. Underpants 200 have leg openings 202 and a band 210 that defines a waist opening 204. Band 210 includes a plurality of O-ring or ring-shaped connectors 220 that are spaced around band 210 in a front, back and side of the undergarment. Each ring-shaped connector 220 is attached to band 210 at two points, such that ring-shaped connectors 220 form a portion of the circumference of waist opening 204. Ring-shaped connector 220 may be made from any a suitable polymeric or metallic material that will provide stability and strength to the connector.

As shown in FIGS. 4 and 4A, which illustrate a top and side view of the magnetic device shown in FIG. 2, magnetic device 208 includes a pair of polarized magnets 212 that are used to create the therapeutic magnetic field. Magnetic device 208 further includes a flexible strap portion 214 with one of the pair of magnets 212 positioned and secured at either end thereof. In an embodiment, strap portion 214 is of a polymeric material that is molded over magnets 212 to secure the magnets therein and to provide a thin, unobtrusive profile to magnetic device 208. In another embodiment as shown in FIG. 6, magnetic device 208 may be formed such that it has a substantially u-shaped profile prior to attachment to the undergarment.

Magnetic device 208 is attached to underpants 200 at ring-shaped connector 220 by magnetic attraction between the pair of magnets 212, such that strap portion 214 of magnetic device 208 encircles a portion of ring-shaped connector 220 and magnets 212 are disposed within ring-shaped connector 220. In various embodiments, underpants 200 may include a plurality of ring-shaped connectors 220 attached to band 210 with one or more connectors 220 having a magnetic device 208 attached thereto.

FIG. 5 is an enlarged view of a band portion of an undergarment in accordance with another embodiment of the present invention. Band 510 is a continuous band, as in the embodiment of FIG. 1, with a ring-shaped connector 520 attached in such a manner that the connector 520 hangs within an aperture 506 that is adjacent band 510. A magnetic device 508 is shown encircling band 510 such that magnets 512 are positioned within ring-shaped connector 520. Alternatively, it should be understood that magnetic device 508 may also be attached solely to ring-shaped connector 520, as in the embodiment of FIG. 2. In another embodiment, ring-shaped connector 520 may hang from band 510 on an interior of the underpants. In such an arrangement an aperture, such as aperture 506, is not necessary for allowing contact between the magnets 512 of magnetic device 508 and a wearer's skin.

A magnet for use in embodiments according to the present invention may be of any shape, such as coin-shaped, square, rectangular, and semicircular, and/or size, for example, having a length or diameter in the range of 0.5 cm to 2.0 cm and a thickness in the range of 1 mm to 5 mm. In addition, magnets for use in embodiments hereof may be made of a rare earth metal, such as neodymium-boron, having a strength above 450 gauss.

Underpants or underwear in accordance with embodiments of the present invention may be of any style as may be worn by a male or female, such as a brief, bikini, hiphugger, boyshort or thong. Further, underpants or underwear in accordance with embodiments of the present invention may be made from any natural or synthetic material, such as cotton, nylon, spandex, silk and/or combinations thereof, which are customarily used in making such garments as would be apparent to one of ordinary skill in the art.

While various embodiments according to the present invention have been described above, it should be understood that they have been presented by way of illustration and example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. An undergarment for applying a magnetic field to a wearer for therapy, the undergarment comprising:
underpants having leg openings and a band defining a waist opening, wherein the underpants include an aperture proximate the band; and
a detachable magnetic device having a pair of magnets for creating the magnetic field, the magnetic device including a strap portion connecting the magnets, wherein the magnetic device is attached to the underpants at the aperture by attraction between the pair of magnets.

2. The undergarment of claim 1, wherein the strap portion of the magnetic device encircles the band of the underpants when the magnetic device is attached to the underpants.

3. The undergarment of claim 2, wherein the magnets of the magnetic device are disposed within the aperture when the magnetic device is attached to the underpants.

4. The undergarment of claim 1, wherein the underpants include a plurality of apertures proximate the band with one or more of the apertures having a magnetic device attached thereto.

5. The undergarment of claim 1, wherein the aperture is a slit and the strap portion of the magnetic device straddles the slit such that one of said pair of magnets is positioned against an interior surface of the underpants and the other of said pair of magnets is positioned against an exterior surface of the underpants when the magnetic device is attached to the underpants.

6. An undergarment for applying a magnetic field to a wearer for therapy, the undergarment comprising:
underpants having leg openings and a band defining a waist opening, wherein the band includes a ring-shaped connector attached thereto; and
a detachable magnetic device having a pair of magnets for creating the magnetic field, the magnetic device including a strap portion between the magnets, wherein the magnetic device is attached to the underpants through the ring-shaped connector by attraction between the pair of magnets.

7. The undergarment of claim 6, wherein the strap portion of the magnetic device encircles a portion of the ring-shaped connector when the magnetic device is attached to the underpants.

8. The undergarment of claim 7, wherein the magnets of the magnetic device are disposed within the ring-shaped connector when the magnetic device is attached to the underpants.

9. The undergarment of claim 6, wherein the underpants include a plurality of ring-shaped connectors attached to the band with one or more connectors having a magnetic device attached thereto.

10. The undergarment of claim 6, wherein the ring-shaped connector forms a portion of the circumference of the waist opening.

11. The undergarment of claim 6, wherein the ring-shaped connector hangs against an interior surface of the underpants.

12. The undergarment of claim 6, further comprising an aperture proximate the band, wherein the ring-shaped connector hangs within the aperture when the magnetic device is attached to the underpants.
